(19) United States
(12) Patent Application Publication
(10) Pub. No.: US 2011/0104142 A1
(43) Pub. Date: May 5, 2011

(54) FORMULATIONS OF PEG-FUNCTIONALISED SERINE PROTEASES WITH HIGH CONCENTRATIONS OF AN AROMATIC PRESERVATIVE

(75) Inventors: Christian Rischel, Copenhagen S (DK); Anders Dybdal, Ballerup (DK); Michael Bech Jensen, Allerod (DK)

(73) Assignee: Novo Nordisk Health Care AG, Zurich (CH)

(21) Appl. No.: 12/990,058

(22) PCT Filed: May 22, 2009
(86) PCT No.: PCT/EP09/56210

§ 371 Date: Jan. 18, 2011

(30) Foreign Application Priority Data
May 23, 2008 (EP) 08104071.9
Nov. 11, 2008 (EP) 08168826.9

(51) Int. Cl. A61K 38/48; A61P 43/00

(52) U.S. Cl. 424/94.64

(57) ABSTRACT

The invention relates to a liquid, aqueous pharmaceutical composition comprising a Factor VII polypeptide (i) functionalised with one or more polyethylene glycol (PEG) moieties, said PEG moieties having a molecular weight of at least 300 Da; a buffering agent (ii) suitable for keeping pH in the range of from about 5.0 to about 9.0; and at least one aromatic preservative (iii) in a concentration of at least 0.1 mg/mL.

### FIELD OF THE INVENTION

The present invention relates to novel formulations of serine proteases (in the following: multiple-dosage pharmaceutical compositions) comprising a serine protease functionalised with one or more polyethylene glycol (PEG) moieties, a buffering agent, and an aromatic preservative.

### BACKGROUND OF THE INVENTION

Blood clotting Factor VIIa (FVIIa) has proven to be an important therapeutic agent for the treatment of blood clotting disorders such as haemophilia A, haemophilia B, Glanzmann's thrombasthenia and Factor VII(a) deficiency. It is also used to enhance blood coagulation in humans that are subject to life-threatening, diffuse or surgically inaccessible bleedings but who otherwise do not have a blood clotting disorder.

The current, commercially available, recombinant Factor VIIa (rFVIIa) formulation NovoSeven® (Novo Nordisk A/S, Denmark), is presented as a vial (about 3.0 mL container volume) containing a freeze-dried cake of 1.2 mg recombinant human Factor VIIa, 5.84 mg NaCl, 2.94 mg CaCl₂, 2H₂O, 2.64 mg GlyGly, 0.14 mg polysorbate 80, and 60.0 mg mannitol. This product is reconstituted to pH 5.5 by 2.0 mL water for injection (WFI) prior to use, thus yielding a concentration of the Factor VIIa of about 0.6 mg/mL.

There are several advantages associated with the use of a preserved, liquid formulation rather than a freeze-dried cake that is reconstituted with WFI immediately prior to injection. One such advantage is that a preserved liquid is more convenient to use. Another advantage of a preserved liquid is that the patient or caregiver may dose several times from the same vial.

For therapeutic applications where administration of larger amounts (e.g. 10-20 mg) of an activated Factor VII polypeptide (e.g. rhFVIIa) is necessary, it is inconvenient to utilize a formulation like the NovoSeven® composition, because a fairly large volume (e.g. 15-30 mL) needs to be administered, There is, therefore, also still a need for a concentrated FVII polypeptide formulation, such that a suitable amount of the FVII polypeptide can be provided in a small volume.

Liquid formulations of serine proteases, such as Factor VII polypeptides, are subject to degradation by autolysis because they themselves are both biological enzymes and substrates. Factors II, VII, IX and X are four such examples of serine proteases. Formulating a protease such as a FVII polypeptide is a major challenge to the pharmaceutical industry because FVII polypeptides readily cleave other FVII polypeptides in the same formulation, rendering them inactive. In liquid formulations, FVII polypeptides auto-inactivate within a period of a few hours and the problem is particularly acute when the concentration of FVII polypeptide is high. Therefore, in creating a liquid formulation of a FVII polypeptide, autolysis is the greatest hurdle to be overcome.

Liquid formulations of Factor VII polypeptides containing Factor VII inhibitors/stabilizers have previously been described. However, these Factor VII inhibitors/stabilizers must be injected together with the Factor VII polypeptide molecule, and the effect of such Factor VII inhibitors/stabilizers on humans is generally not known.

WO 2005/002615 A1 discloses a liquid, aqueous pharmaceutical composition comprising a Factor VII polypeptide; a buffering agent suitable for keeping pH in the range of from about 5.0 to about 9.0; at least one metal-containing agent, wherein said metal is selected from the group consisting of a first transition series metal of oxidation state+II, except zinc; and a non-ionic surfactant.

WO 2005/016365 A1 discloses a liquid, aqueous pharmaceutical composition comprising at least 0.01 mg/mL of a Factor VII polypeptide (i); a buffering agent (ii) suitable for keeping pH in the range of from about 5.0 to about 9.0; and at least one stabilising agent (iii) comprising a —C(═N—Z¹—R¹)—NH—Z²—R² motif (e.g. a benzamidine or an arginine).

Multiple-dosage formulations are advantageous for injectable pharmaceutical products. If several injection dosages are retrieved from the same vial over several days, a preservative is often a requirement from the regulatory authorities. A number of different compounds have been used as preservatives in injectable products (S. Nema, N. R. Washkuhn and R. J. Brendel: Excipients and their use in injectable products, PDA Journal of Pharmaceutical Science and Technology 51 (4), 166-171).

However, the presence of a preservative will often reduce the solubility of a Factor VII polypeptide, another major problem associated with the formulation of such a serine protease in a liquid. There is a need for novel multiple-dosage liquid pharmaceutical compositions comprising an activated Factor VII polypeptide in a relatively high concentration, together with a preservative.

### SUMMARY OF THE INVENTION

The invention relates to the creation of a soluble FVII polypeptide with improved stability in solution.

The invention also relates to the creation of a stabilised FVII polypeptide that has improved solubility in a liquid solution. The inventor has created a FVII polypeptide liquid formulation, which is suitable for retraction of multiple-doses. Such a formulation can be obtained by combining an aromatic preservative with a Factor VII polypeptide which is functionalized with one or more polyethylene glycol moieties.

In a first aspect, the invention relates to a liquid, aqueous pharmaceutical composition comprising:

(i) a Factor VII(a) polypeptide that is functionalised with one or more polyethylene glycol (PEG) moieties, said PEG moieties having a molecular weight of at least 300 Da;

(ii) a buffering agent that is suitable for keeping pH in the range of from about 5.0 to about 9.0; and

(iii) at least one aromatic preservative, in a concentration of at least 0.1 mg/mL.

More specifically, the invention relates to a liquid, aqueous pharmaceutical composition comprising:

(i) a Factor VII(a) polypeptide functionalised with one or more polyethylene glycol (PEG) moieties, said PEG moieties having a molecular weight of 5,000-50,000 Da;

(ii) a buffering agent suitable for keeping pH in the range of from about 5.0 to about 9.0; and

(iii) at least one aromatic preservative in a concentration of at least 0.1 mg/mL.

A second aspect of the invention relates to a liquid, aqueous pharmaceutical composition as defined herein for use as a medicament.

A third aspect of the invention relates to the use of a liquid, aqueous pharmaceutical composition as defined herein for the preparation of a medicament for treating a Factor VII(a)-responsive disorder.

A fourth aspect of the invention relates to a method for treating a Factor VII(a)-responsive disorder, the method comprising administering to a subject in need thereof an effective amount of a liquid, aqueous pharmaceutical composition as defined herein.

A fifth aspect of the invention relates to an air-tight container containing a liquid, aqueous pharmaceutical composition as defined herein, and, optionally, an inert gas.

A sixth aspect of the invention relates to a kit for the preparation of the composition as defined herein, said kit comprising:

(a) a first container comprising at least the Factor VII polypeptide (i) in freeze-dried form;

(b) a second container comprising an aqueous reconstitution liquid, said liquid at least comprising the at least one aromatic preservative (ii).

### BRIEF DESCRIPTION OF THE FIGURES

FIG. 1 illustrates that in solutions containing rFVIIa and m-cresol, rFVIIa tends to precipitate more as the concentration of m-cresol is increased from 0-3 mg/ml. FIG. 1 also illustrates that, in [otherwise identical] solutions of 10K-PEG-rFVIIa and m-cresol, 10K-PEG-rFVIIa precipitates very little to not at all as the concentration of m-cresol is increased from 0-3 mg/ml. Furthermore, FIG. 1 shows that, in solutions containing 6 mg/ml phenol, 10K-PEG-rFVIIa is more soluble (precipitates less) than rFVIIa.

### DETAILED DESCRIPTION OF THE INVENTION

As mentioned above, the present invention resides in the development of a novel stabilised liquid, aqueous pharmaceutical composition comprising a high concentration of a Factor VII polypeptide functionalised with one or more polyethylene glycol (PEG) moieties together with a relatively high concentration of an aromatic preservative.

More specifically, the liquid, aqueous pharmaceutical composition comprises:

(i) a Factor VII polypeptide that is functionalised with one or more polyethylene glycol (PEG) moieties, said PEG moieties having a molecular weight of at least 300 Da;

(ii) a buffering agent that is suitable for keeping pH in the range of from about 5.0 to about 9.0; and

(iii) at least one aromatic preservative, in a concentration of at least 0.1 mg/mL.

Factor VII Polypeptide (i) Functionalised with PEG Moieties

Factor VII Polypeptide

The biological effect of the pharmaceutical composition is mainly ascribed to the presence of the Factor VII polypeptide, although other active ingredients may be included in combination with the Factor VII polypeptide.

As used herein, the term “Factor VII polypeptide” encompasses wild-type Factor VII (i.e. a polypeptide having the amino acid sequence disclosed in U.S. Pat. No. 4,784,950), as well as variants of Factor VII exhibiting substantially the same or improved biological activity relative to wild-type Factor VII. The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa. The term “Factor VII polypeptide” also encompasses polypeptides, including variants, in which the Factor VIIa biological activity has been substantially modified, increased or reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

The biological activity of Factor VIIa in blood clotting derives from its ability to (1) bind to Tissue Factor (TF) and (2) catalyze the proteolytic cleavage of Factor IX or Factor X to produce activated Factor IX or X (Factor IXa or Xa, respectively).

For the purposes of assessing the success of the invention, biological activity of the liquid formulated Factor VII polypeptides (“Factor VII biological activity”) may be quantified by measuring the ability of a preparation to promote blood clotting, cf. Assay 4 described herein. In this assay, biological activity is expressed as the reduction in clotting time relative to a control sample and is converted to “Factor VII units” by comparison with a pooled human serum standard containing 1 unit/mL Factor VII activity. Alternatively, Factor VIIa biological activity may be quantified by (i) measuring the ability of Factor VIIa or a Factor VII-related polypeptide to produce activated Factor X (Factor Xa) in a system comprising TF embedded in a lipid membrane and Factor X (Persson et al., J. Biol. Chem. 272:19919-19924, 1997); (ii) measuring Factor X hydrolysis in an aqueous system (“In Vitro Proteolysis Assay”, see Assay 2 below); (iii) measuring the physical binding of Factor VIIa or a Factor VII-related polypeptide to TF using an instrument based on surface plasmon resonance (Persson, FEBS Letts. 413:359-363, 1997); (iv) measuring hydrolysis of a synthetic substrate by Factor VIIa and/or a Factor VII-related polypeptide (“In Vitro Hydrolysis Assay”, see Assay 1 below); or (v) measuring generation of thrombin in a TF-independent in vitro system (see Assay 3 below).

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay (Assay 4), proteolysis assay (Assay 2), or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor VIIa that has been produced in the same cell type when tested in one or more of a clotting assay (Assay 4), proteolysis assay (Assay 2), or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, polypeptides having an amino acid sequence that differs from the sequence of wild-type Factor VII by insertion, deletion, or substitution of one or more amino acids.

Non-limiting examples of Factor VII variants having substantially the same biological activity as wild-type Factor VII include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189; and FVII variants exhibiting increased proteolytic stability as disclosed in WO 02/38162 (Scripps Research Institute); FVII variants having a modified Gla-domain and exhibiting an enhanced membrane binding as disclosed in WO 99/20767 (University of Minnesota); and FVII variants as disclosed in WO 01/58935 (Maxygen ApS).

Non-limiting examples of Factor VII variants having increased biological activity compared to wild-type FVIIa include FVII variants as disclosed in WO 01/83725, WO 02/22776, WO 02/077218, WO 03/27147, WO 03/37932; WO 02/38162 (Scripps Research Institute); and FVIIa variants with enhanced activity as disclosed in JP 2001061479 (Chemo-Sero-Therapeutic Res Inst.).

Non-limiting examples of Factor VII variants having substantially reduced or modified biological activity relative to wild-type Factor VII include R152E-FVIIa (Wildgoose et al., Biochem 29:3413-3420, 1990), S344A-FVIIa (Kazama et al., J. Biol. Chem. 270:66-72, 1995), FFR-FVIIa (Hoist et al., Eur. J. Vasc. Endovasc. Surg. 15:515-520, 1998) and Factor VIIa lacking the Gla domain (Nicolaisen et al., FEBS Letts. 317:245-249, 1993).

Examples of Factor VII polypeptides include, without limitation, wild-type Factor VII, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, M298Q/K337A-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII;R152E-Factor VII, S344A-Factor VII, Factor VIIa lacking the Gla domain; and P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys, and FVII having substitutions, deletions, or additions in the amino acid sequence Ile153-Arg223.

In some embodiments, the Factor VII polypeptide is human Factor VIIa (hFVIIa), preferably recombinant human Factor VIIa (rhVIIa).

In other embodiments, the Factor VII polypeptide is a Factor VII sequence variant.

In some embodiments, the Factor VII polypeptide has a glycosylation different from wild-type human Factor VII.

In the currently most interesting embodiment, the protein is a Factor VII polypeptide in its activated form.

In various embodiments, e.g. those where the Factor VII polypeptide is a Factor VII-related polypeptide or a Factor VII sequence variant, the ratio between the activity of the Factor VII polypeptide and the activity of native human Factor VIIa (wild-type FVIIa) is at least about 1.25, preferably at least about 2.0, or 4.0, most preferred at least about 8.0, when tested in the In Vitro Proteolysis Assay” (Assay 2) as described in the present specification.

In some embodiments, the Factor VII polypeptides are Factor VII-related polypeptides, in particular variants, wherein the ratio between the activity of said Factor VII polypeptide and the activity of native human Factor VIIa (wild-type FVIIa) is at least about 1.25 when tested in the In Vitro Hydrolysis Assay” (see Assay 1 below); in other embodiments, the ratio is at least about 2.0; in further embodiments, the ratio is at least about 4.0.

In a pharmaceutical composition, it is often desirable that the concentration of the active ingredient is such that the application of a unit dose does not cause unnecessary discomfort to the patient. Thus, a unit dose of more than about 2-10 mL is often undesirable. For the purpose of the present invention, the concentration of the Factor VII polypeptide is therefore typically relatively high, i.e. at least 0.1 mg/mL. In different embodiments, the Factor VII polypeptide is present in a concentration of 0.1-90 mg/mL; 0.5-80 mg/mL; 1.0-80 mg/mL; 1.5-70 mg/mL; 2-60 mg/mL; 3-50 mg/mL; or 5-50 mg/mL; or 10-50 mg/ml; or 15-50 mg/ml.

Factor VIIa concentration is conveniently expressed as mg/mL or as IU/mL, with 1 mg usually representing 43,000-56,000 IU for unmodified rFVIIa. (The specific activity may be lowered for pegylated Factor VIIa).

The Factor VII polypeptide is typically represented in a glycoform wherein one or more oligonucleotides are covalently linked to (an) amino acid(s) of the polypeptide chain, most typically asparagine-linked (N-linked) or serine-linked (O-linked). The naturally occurring glycosylation sites of Factor VII are at positions Asn-145 (N145), Asn-322 (N322), Ser-52 (S52), and Ser-60 (S60).

PEG Moieties

The term “functionalised with a PEG moiety” is in terms of the present invention synonymous with the term “PEGylated”. The one or more PEG moieties which represent the functionalisation of the Factor VII polypeptide are covalently linked either to any part of the polypeptide backbone of the Factor VII polypeptide or to an oligosaccharide which is an integral part of the Factor VII polypeptide (“glycopegylated Factor VII polypeptide”). Glycopegylated Factor VII is thoroughly described in the applicant's earlier applications WO 2004/000366 A1 and WO 2005/014035 A1. This being said, the PEG moieties typically have a molecular weight of at least 300 Da, such as 300-100,000 Da; such as about 5,000-50,000 Da; such as about 10,000 to about 45,000 Da; such as about 35,000 to about 45,000; such as about 39,000 to 42,000 Da, such as about 40,000 to about 41,000 Da; such as about 500-20,000 Da, or 500-15,000 Da, or 2,000-15,000 Da, or 3,000-15,000 Da, or 3,000-12,000 Da, or about 10 Da. The PEG moieties may be linear or branched. The term 40K refers to a PEG moiety which is approximately 40,000 to 41,000 Da.

As will be evident for the person skilled in the art, the PEG moiety may need an “attachment group” in order for the PEG moiety to be attached to the Factor VII polypeptide as outlined above.

The term “attachment group” is intended to indicate a functional group of the oligosaccharide moiety capable of attaching a polymer molecule. Useful attachment groups are, for example, amine, hydroxyl, carboxyl, aldehyde, ketone, sulfhydryl, succinimidyl, maleimide, vinyl sulfone or haloacetate.

The attachment group on the oligosaccharide moiety may be activated before reaction with the polymer. Alternatively, a group present on the polymer may be activated before reaction with the oligosaccharide moiety. The activated group, whether present on the oligosaccharide- or polymer moiety may be in the form of an activated leaving group. The term activated leaving group includes those moieties which are easily displaced in organic- or enzyme-regulated substitution reactions. Activated leaving groups are known in the art, see, for example, Vocadlo et al., In Carbohydrate Chemistry and Biology, Vol 2, Wiley-VCH Verlag, Germany (2000); Kodama et al., Tetrahedron Letters 34:6419 (1993); Lougheed et al., J. Biol. Chem. 274:37717 (1999).

Methods and chemistry for activation of polymers are described in the literature. Commonly used methods for activation of polymers include activation of functional groups with cyanogen bromide, periodate, glutaraldehyde, biepoxides, epichlorohydrin, divinylsulfone, carbodiimide, sulfonyl halides, trichlorotriazine, etc. (see, for example, Taylor (1991), Protein immobilization, Fundamentals and Applications, Marcel Dekker, N.Y.; Wong (1992), Chemistry of protein Conjugation and Crosslinking, CRC Press, Boca Raton; Hermanson et al., (1993), Immobilized Affinity Ligand Techniques, Academic Press, N.Y.; Dunn et al., Eds. Polymeric Drugs and Drug Delivery Systems, ACS Symposium Series Vol. 469, American Chemical Society, 1991.)

Reactive groups and classes of reactions useful in practicing the present invention are generally those which proceed under relatively mild conditions. These include, but are not limited to nucleophilic substitutions (e.g., reaction of amines and alcohols with acyl halides, active esters), electrophilic substitutions (e.g., enamine reactions) and additions to carbon-carbon and carbon-heteroatom bonds (e.g., Michael reaction, Diels-Alder addition).

These and other useful reactions are described in, for example, March, Advanced Organic Chemistry, 3rd edition, John Wiley & Sons, N.Y. 1985; Hermanson, Bioconjugate Techniques, Academic Press, San Diego, 1996; Feeney et al, Modifications of Proteins, Advances in Chemistry Series, Vol. 198, American Chemical Society, 1982.

The reactive functional groups can be chosen such that they do not participate in, or interfere with, the reactions necessary to assemble the oligosaccharide and the polymer moiety. Alternatively, a reactive functional group can be protected from participating in the reaction by the presence of a protective group. For examples of useful protecting groups, see, for example, Greene et al., Protective groups in Organic Synthesis, John Wiley & Sons, N.Y., 1991.

General approaches for linking carbohydrates to other molecules are known in the literature (see, e.g., Lee et al., Biochemistry 28:1856 (1989); Bhatia et al., Anal. Biochem. 178:408 (1989); Janda et al., J. Am. Chem. Soc. 112:8886 (1990); and Bednarski et al., WO 92/18135.

Buffering Agent (ii)

In order to render the liquid, aqueous pharmaceutical composition useful for direct parenteral administration to a mammal such as a human, it is normally required that the pH value of the composition is held within reasonable limits, such as from about 5.0 to about 9.0. To ensure a suitable pH value under the conditions given, the pharmaceutical composition also comprises a buffering agent (ii) suitable for keeping pH in the range of from about 5.0 to about 9.0.

The term “buffering agent” encompasses those agents, or combinations of agents, which maintain the solution pH in an acceptable range from about 5.0 to about 9.0.

In one embodiment, the buffering agent (ii) is at least one component selected from the groups consisting of acids and salts of MES, PIPES, ACES, BES, TES, HEPES, TRIS, histidine, imidazole, glycine, glycylglycine, glycinamide, phosphoric acid, acetic acid (e.g. sodium or calcium acetate), lactic acid, glutaric acid, citric acid, tartaric acid, malic acid, maleic acid and succinic acid. It is to be understood that the buffering agent may comprise a mixture of two or more components, wherein the mixture is able to provide a pH value in the specified range. Examples of such buffers are acetic acid and sodium acetate.

The concentration of the buffering agent is chosen so as to maintain the preferred pH of the solution. In various embodiments, the concentration of the buffering agent is 1-100 mM; such as 1-50 mM; such as 1-25 mM; or 2-20 mM.

In one embodiment, the pH of the composition is kept from about 5.0 to about 8.0; such as from about 5.0 to about 7.5; from about 5.0 and about 7.0; from about 5.0 to about 6.5, from about 5.0 to about 6.0, from about 5.5 to about 7.0; from about 5.5 to about 6.5, from about 6.0 to about 7.0, from about 6.4 to about 6.6, or from about 5.2 to about 5.7.

Aromatic Preservative(s) (iii)

The pharmaceutical composition further comprises at least one aromatic preservative (iii) in a concentration of at least 0.1 mg/mL.

Preservatives are typically included in a composition to retard microbial growth; that is, the aromatic preservative has bacteriostatic or bacteriocidal effects. However, the inventor finds that aromatic preservative(s), in combination with antioxidant(s), also have a very pronounced effect on the stability of Factor VII polypeptides in aqueous solution, in particular those that are formulated at fairly high concentrations.

In the following, the term “aromatic” refers to chemical compounds containing in their structure a 6-membered unsaturated ring of carbon atoms, that is, a benzene ring.

Examples of aromatic preservatives of the invention include phenol, benzyl alcohol, orto-cresol, meta-cresol, para-cresol, chloro-cresol, methyl paraben, propyl paraben, benzalkonium chloride, and benzethonium chloride.

In one embodiment of the invention, the at least one aromatic preservative (iii) is meta-cresol and/or phenol and/or benzyl alcohol and/or chlorocresol.

The at least one aromatic preservative (iii) is normally included at a concentration of 0.1-30.0 mg/Ml, such as 0.1-20.0 mg/mL, depending on the pH range and type of aromatic preservative. For example, typical concentrations are 1.0-5.0 mg/mL, such as 1.0-4.0 mg/mL meta-cresol; 1.0-10.0 mg/mL, such as 1-6 mg/mL phenol; 5.0-30.0 mg/mL, such as 5.0-20.0 mg/mL benzyl alcohol; or 1.0-5.0 mg/mL, such as 1.0-3.0 mg/mL chlorocresol.

Antioxidant(s) (iv)

It has been found that the stability of the Factor VII polypeptide in the aqueous composition can be further increased by combining the aromatic preservative(s) (iii) with an antioxidant(s) (iv). The at least one antioxidant is typically present in a concentration of at least 0.1 mg/mL.

In different embodiments, the at least one antioxidant (iv) is selected from the group consisting of L-methionine, D-methionine, methionine analogues, methionine-containing peptides, methionine-homologues, ascorbic acid, cysteine, homocysteine, gluthatione, cystine and cysstathionine. In a preferred embodiment, the antioxidant is L-methionine.

The concentration of the at least one antioxidant is typically 0.1-5.0 mg/mL, such as 0.1-4.0 mg/mL, 0.1-3.0 mg/mL, 0.1-2.0 mg/mL, or 0.5-2.0 mg/mL.

Further Components

The liquid, aqueous pharmaceutical composition may, in addition to the before-mentioned components, comprise additional components beneficial for the preparation, formulation, or administration of the composition.

In some embodiments, the composition further comprises a tonicity modifying agent (v).

As used herein, the term “tonicity modifying agent” includes agents which contribute to the osmolality of the solution. The tonicity modifying agent (v) includes at least one agent selected from the group consisting of neutral salts, amino acids, peptides of 2-5 amino acid residues, monosaccharides, disaccharides, polysaccharides, and sugar alcohols. In some embodiments, the composition comprises two or more of such agents in combination.

By “neutral salt” is meant a salt that is neither an acid nor a base when dissolved in an aqueous solution.

In one embodiment, at least one tonicity modifying agent (v) is a neutral salt selected from the groups consisting of sodium salts, potassium salts, calcium salts, and magnesium salts, such as sodium chloride, potassium chloride, calcium chloride, calcium acetate, calcium gluconate, calcium laevulate, magnesium chloride, magnesium acetate, magnesium gluconate, and magnesium laevulate.

In a further embodiment, the tonicity modifying agent (v) includes sodium chloride in combination with at least one selected from the groups consisting of calcium chloride, calcium acetate, magnesium chloride and magnesium acetate.

In a still further embodiment, the tonicity modifying agent (v) is at least one selected from the group consisting of sodium chloride, calcium chloride, sucrose, glucose, and mannitol.

In different embodiments, the tonicity modifying agent (v) is present in a concentration of at least 1 mM, at least 5 mM, at least 10 mM, at least 20 mM, at least 50 mM, at least 100 mM, at least 200 mM, at least 400 mM, at least 800 mM, at least 1000 mM, at least 1200 mM, at least 1500 mM, at least 1800 mM, at least 2000 mM, or at least 2200 mM.

In one series of embodiments, the tonicity modifying agent (v) is present in a concentration of 5-2200 mM, such as 25-2200 mM, 50-2200 mM, 100-2200 mM, 200-2200 mM, 400-2200 mM, 600-2200 mM, 800-2200 mM, 1000-2200 mM, 1200-2200 mM, 1400-2200 mM, 1600-2200 mM, 1800-2200 mM, or 2000-2200 mM; 5-1800 mM, 25-1800 mM, 50-1800 mM, 100-1800 mM, 200-1800 mM, 400-1800 mM, 600-1800 mM, 800-1800 mM, 1000-1800 mM, 1200-1800 mM, 1400-1800 mM, 1600-1800 mM; 5-1500 mM, 25-1400 mM, 50-1500 mM, 100-1500 mM, 200-1500 mM, 400-1500 mM, 600-1500 mM, 800-1500 mM, 1000-1500 mM, 1200-1500 mM; 5-1200 mM, 25-1200 mM, 50-1200 mM, 100-1200 mM, 200-1200 mM, 400-1200 mM, 600-1200 mM, or 800-1200 mM.

In a preferred embodiment of the invention, at least one tonicity modifying agent (v) is an ionic strength modifying agent (v/a).

As used herein, the term “ionic strength modifying agent” includes agents which contribute to the ionic strength of the solution. The agents include, but are not limited to, neutral salts, amino acids, peptides of 2 to 5 amino acid residues. In some embodiments, the composition comprises two or more of such agents in combination.

Preferred examples of ionic strength modifying agents (v/a) are neutral salts such as sodium chloride, potassium chloride, calcium chloride and magnesium chloride. A preferred agent (v/a) is sodium chloride.

The term “ionic strength” is the ionic strength of the solution (p) which is defined by the equation: μ=½Σ([i](Zᵢ²)), where μ is the ionic strength, [i] is the millimolar concentration of an ion, and Zᵢ is the charge (+ or −) of that ion “(see, e.g., Solomon, Journal of Chemical Education, 78(12):1691-92, 2001; James Fritz and George Schenk: Quantitative Analytical Chemistry, 1979).

In different embodiments of the invention, the ionic strength of the composition is at least 50, such as at least 75, at least 100, at least 150, at least 200, at least 250, at least 400, at least 500, at least 650, at least 800, at least 1000, at least 1200, at least 1600, at least 2000, at least 2400, at least 2800, or at least 3200.

In some specific embodiments, the total concentration of the tonicity modifying agent (v) and the ionic strength modifying agent (v/a) is in the range of 1-500 mM, such as 1-300 mM, or 10-200 mM, or 20-150 mM, depending on the effect any other ingredients may have on the tonicity and ionic strength.

In one embodiment, the composition is isotonic; in another, it is hypertonic.

The term “isotonic” means “isotonic with serum”, i.e. at about 300±50 milliosmol/kg. The tonicity is meant to be a measure of osmolality of the solution prior to administration. The term “hypertonic” is meant to designate levels of osmolality above the physiological level of serum, such as levels above 300±50 milliosmol/kg.

Also, a particular embodiment of the present invention relates to the combination of the aromatic preservative(s) (iii) and antioxidant(s) (iv) with a fairly high concentration of an ionic strength modifying agent (v/a) selected from the group consisting of sodium salts, calcium salts and magnesium salts. In this embodiment, the ionic strength modifying agent (v/a), i.e. the sodium salt, calcium salt and/or magnesium salt, is present in a concentration of 15-1000 mM, such as 25-1000 mM, 50-1000 mM, 100-1000 mM, 200-1000 mM, 300-1000 mM, 400-1000 mM, 500-1000 mM, 600-1000 mM, 700-1000 mM; 15-800 mM, 25-800 mM, 50-800 mM, 100-800 mM, 200-800 mM, 300-800 mM, 400-800 mM, 500-800 mM; 15-600 mM, 25-600 mM, 50-600 mM, 100-600 mM, 200-600 mM, 300-600 mM; 15-400 mM, 25-400 mM, 50-400 mM, or 100-400 mM.

Within these embodiments, the sodium salt may be sodium chloride, the calcium salt may be selected from the group consisting of calcium chloride, calcium acetate, calcium gluconate, and calcium laevulate, and the magnesium salt may be selected from the group consisting of magnesium chloride, magnesium acetate, magnesium gluconate, magnesium laevulate, and magnesium salts of strong acids. In a more specific embodiment, a calcium salt and/or a magnesium salt is/are used in combination with sodium chloride.

In one currently preferred embodiment, the composition comprises one or more ionic strength modifying agents selected from the group consisting of calcium (Ca²⁺) salts and magnesium (Mg²⁺) salts, e.g. one or more salts selected from the group consisting of calcium chloride, calcium acetate, calcium gluconate, calcium laevulate, magnesium chloride, magnesium acetate, magnesium sulphate, magnesium gluconate, magnesium laevulate, magnesium salts of strong acids. In one embodiment hereof, the concentration of the calcium (Ca²⁺) and/or magnesium (Mg²⁺) salt(s) is at least 2 mM, such as at least 5 mM or about 10 mM.

In further embodiment, which may be combined with the foregoing, the pharmaceutical composition may also include a non-ionic surfactant (vi). Surfactants (also known as detergents) generally include those agents which protect the protein from air/solution interface induced stresses and solution/surface induced stresses (e.g. resulting in protein aggregation).

Typical types of non-ionic surfactants are polysorbates, poloxamers, polyoxyethylene alkyl ethers, polyethylene/polypropylene block co-polymers, polyethyleneglycol (PEG), polyxyethylene stearates, and polyoxyethylene castor oils.

Illustrative examples of non-ionic surfactants are Tween®, polysorbate 20, polysorbate 80, Brij-35 (polyoxyethylene dodecyl ether), poloxamer 188, poloxamer 407, PEG8000, Pluronic® polyols, polyoxy-23-lauryl ether, Myrj 49, and Cremophor A, in particular poloxamer 188.

In one embodiment, the non-ionic surfactant is present in an amount of 0.005-2.0% by weight.

Although a combination of the preservative(s) and the antioxidant(s) is believed to dramatically reduce the need for any further stabilising agents, such agents may in principle be added if desired. Examples of such further stabilising agents are those selected from (a) metal-containing agents, wherein said metal is selected from the group consisting of first transition series metals of oxidation state+II, except zinc; and (b) stabilising agent comprising a —C(═N—Z¹—R¹)—NH—Z²—R² motif.

With respect to the stabilising agents of type (a), these are described and defined in WO 2005/002615.

With respect to the stabilising agents of type (b), these are described and defined (in general and explicitly with respect to the detailed meaning of Z¹, Z², R¹ and R²) in WO 2005/016365. For convenience it should although be mentioned that Z¹ and Z² independently are selected from the group consisting of —O—, —S—, —NR^{H}— and a single bond, where R^{H} is selected from the group consisting of hydrogen, C₁₋₄-alkyl, aryl and arylmethyl, and R¹ and R² independently are selected from the group consisting of hydrogen, optionally substituted C₁₋₆-alkyl, optionally substituted C₂₋₆-alkenyl, optionally substituted aryl, optionally substituted heterocyclyl, or Z² and R² are as defined above and —C═N—Z¹—R¹ forms part of a heterocyclic ring, or Z¹ and R¹ are as defined above and —C—NH—Z²—R² forms part of a heterocyclic ring, or —C(═N—Z¹—R¹)—NH—Z²—R² forms a heterocyclic ring wherein —Z¹—R¹—R²—Z²— is a biradical.

In a currently preferred embodiment, however, none of said further components of the composition are Factor VII polypeptide stabilizing agents.

### Preferred Embodiment

The present inventors have presently identified the following embodiment as particularly advantageous, namely the liquid, aqueous pharmaceutical composition as defined herein, which comprises:

(i) 1-90 mg/mL of a Factor VII polypeptide functionalised with one or more polyethylene glycol (PEG) moieties, said PEG moieties having a molecular weight of 500-60,000 Da;

(ii) a buffering agent suitable for keeping pH in the range of from about 5.0 to about 9.0;

(iii) at least one aromatic preservative in a concentration of 0.1-20 mg/mL; and

(iv) at least one antioxidant in a concentration of 0.1-5.0 mg/mL.

In another preferred embodiment, the liquid, aqueous composition comprises:

(i) 40K-PEG-rFVIIa,

(ii) a buffering agent which keeps the pH within the range of about 5 to about 6 and

(iii) either phenol in a concentration of 1.0-10.0 mg/ml or m-cresol in a concentration of 1.0-5.0 mg/mL.

In a third preferred embodiment, the liquid, aqueous composition comprises

(i) 40K-PEG-rFVIIa,

(ii) a buffering agent which keeps the pH within the range of about 5 to about 6 and

(iii) a combination of phenol and m-cresol.

Stability

In one embodiment, the compositions according to the present invention are useful as stable ready-to-use liquid compositions of Factor VII polypeptides. The ready-to-use liquid compositions should typically be stable for at least six months, and preferably up to 36 months, when stored at temperatures ranging from 2° C. to 8° C. In another embodiment, the compositions according to the present invention are useful as dry compositions reconstituted with an aqueous liquid prior to use, said liquid containing the aromatic preservative. The freeze-dried composition should typically be stable for at least six months, and preferably up to 36 months, when stored at 25° C. The liquid composition obtained by mixing the dry composition with the reconstitution liquid should typically be stable for at least one week, and preferably up to 4 weeks or longer, when stored at temperatures ranging from 2° C. to 8° C.

The term “stable” is intended to denote that (i) after storage for 6 months at 2° C. to 8° C. the composition retains at least 50% of its initial biological activity as measured by a one-stage clot assay (Assay 4), or (ii) after storage for 6 months at 2° C. to 8° C., the content of heavy chain degradation products is at the most 40% (w/w) assuming that the initial sample comprises no heavy chain degradation products (i.e. only the Factor VII polypeptide is entered into the calculation of the percentage). Preferably, the composition retains at least 70%, such as at least 80%, or at least 85%, or at least 90%, or at least 95%, of its initial activity after storage for 6 months at 2 to 8° C. Also preferably, the content of heavy chain degradation products in the composition is at the most 30% (w/w), at the most 25% (w/w), at the most 20% (w/w), at the most 15% (w/w), at the most 10% (w/w), at the most 5% (w/w), or at the most 3% (w/w).

Preferably, the stable composition retains at least 70%, such as at least 80%, or at least 85%, or at least 90%, or at least 95%, of its initial activity after storage for 6 months at 2 to 8° C.

Preferably, in various embodiments the content of heavy chain degradation products in stable compositions is at the most 30% (w/w), at the most 25% (w/w), at the most 20% (w/w), at the most 15% (w/w), at the most 10% (w/w), at the most 5% (w/w), or at the most 3% (w/w).

Methods of Use

As will be understood, the liquid, aqueous pharmaceutical compositions defined herein can be used in the field of medicine. Thus, the present invention provides the liquid, aqueous pharmaceutical compositions defined herein for use as a medicament, particularly use as a medicament for treating a Factor VII(a)-responsive disorder.

Consequently, the present invention also provides the use of the liquid, aqueous pharmaceutical composition as defined herein for the preparation of a medicament for treating a Factor VII(a)-responsive disorder, as well as a method for treating a Factor VII(a)-responsive disorder, the method comprising administering to a subject in need thereof an effective amount of the liquid, aqueous pharmaceutical composition as defined herein.

The preparations of the present invention may be used to treat any Factor VII-responsive disorder, such as, e.g., bleeding disorders, including those caused by clotting Factor deficiencies (e.g., haemophilia A, haemophilia B, coagulation Factor XI deficiency, coagulation Factor VII deficiency); by thrombocytopenia or von Willebrand's disease, or by clotting Factor inhibitors; as well as intracerebral haemorrhage, or excessive bleeding from any cause. The preparations may also be administered to humans in association with surgery or other trauma or to patients receiving anticoagulant therapy.

The term “effective amount” is the dose to be determined by a qualified practitioner, who may adjust doses in order to achieve the desired response. Factors for consideration of dose will include potency, bioavailability, desired pharmacokinetic/pharmacodynamic profiles, condition of treatment, patient-related factors (e.g. weight, health, age, etc.), presence of co-administered medications (e.g., anticoagulants), time of administration, or other factors known to a medical practitioner.

The term “treatment” is defined as the management and care of a subject, e.g. a mammal, in particular a human, for the purpose of combating the disease, condition, or disorder and includes the administration of a Factor VII polypeptide to prevent the onset of the symptoms or complications, or alleviating the symptoms or complications, or eliminating the disease, condition, or disorder. Pharmaceutical compositions according to the present invention containing a Factor VII polypeptide may be administered parenterally to subjects in need of such a treatment. Non-exclusive examples of such parenteral administration are subcutaneous, intramuscular or intravenous injection, optionally by means of a pen-like device or an infusion pump.

In important embodiments, the pharmaceutical composition is adapted to subcutaneous, intramuscular or intravenous injection according to methods known in the art.

Air-Tight Container

Thus, the present invention also provides an air-tight container (e.g. a vial or a cartridge (such as a cartridge for a pen applicator)) containing a liquid, aqueous pharmaceutical composition as defined herein, and, optionally, an inert gas.

The inert gas may be selected from the groups consisting of nitrogen, argon, etc. The container (e.g. vial or cartridge) is typically made of glass or plastic, in particular glass, optionally closed by a rubber septum or other closure means allowing for needle penetration with preservation of the integrity of the pharmaceutical composition. In a further embodiment, the container is a vial or cartridge enclosed in a sealed bag, e.g. a sealed plastic bag, such as a laminated (e.g. metal (such as aluminium) laminated plastic bag).

A Kit Comprising a Freeze-Dried Factor VII Polypeptide

The above defined liquid, aqueous pharmaceutical composition is mainly intended for direct use, typically for parenteral administration, e.g. by injection. It is envisaged that the liquid, aqueous pharmaceutical composition may be prepared from the corresponding freeze-dried formulation some time before the actual parenteral use by the practitioner or the end-user, e.g. 1-24 hours before use, or even some weeks, e.g. 2-4 weeks before use, for example in the form of a multiple dose batch. In such instances it is convenient for the practitioner or end-user to receive the Factor VII polypeptide in freeze-dried form together with the suitable amount of aqueous reconstitution liquid.

Hence, a further aspect of the present invention relates to kit for the preparation of the composition as defined herein, said kit comprising:

(a) a first container comprising at least the Factor VII polypeptide (i) functionalised with one or more polyethylene glycol (PEG) moieties in freeze-dried form;

(b) a second container comprising an aqueous reconstitution liquid, said liquid at least comprising the buffering agent (ii) and the at least one aromatic preservative (iii).

In some embodiments, the first container and the second container may be arranged as separate compartment of a device, e.g. an ampoule for a syringe device, e.g. a pen.

### EXAMPLES

General Methods

Percentages are (weight/weight) both when referring to solids dissolved in solution and liquids mixed into solutions. For example, Poloxamer 188 refers to the weight of 100% stock/weight of solution.

Assays Suitable for Determining Biological Activity of Factor VII Polypeptides

Factor VII polypeptides useful in accordance with the present invention may be selected by the following suitable assays that can be performed as simple preliminary in vitro tests, namely, the 1^{st} generation clot assay, the in vitro hydrolysis assay, the thrombin generation assay, the one-stage coagulation assay and the Factor X generation assay. Values may be compared to those of wild type FVIIa.

In Vitro Hydrolysis Assay (Assay 1)

The in vitro hydrolysis assay is used to assess the ability of Factor VIIa polypeptides to cleave another peptide or protein.

Native (wild-type) Factor VIIa and Factor VII polypeptide (both hereinafter referred to as “Factor VIIa”) may be assayed for specific activities. They may also be assayed in parallel to directly compare their specific activities. The assay is carried out in a microtiter plate (MaxiSorp, Nunc, Denmark). The chromogenic substrate D-Ile-Pro-Arg-p-nitroanilide (S-2288, Chromogenix, Sweden), final concentration 1 mM, is added to Factor VIIa (final concentration 100 nM) in 50 mM HEPES, pH 7.4, containing 0.1 M NaCl, 5 mM CaCl₂ and 1 mg/mL bovine serum albumin. The absorbance at 405 nm is measured continuously in a SpectraMax™ 340 plate reader (Molecular Devices, USA). The absorbance developed during a 20-minute incubation, after subtraction of the absorbance in a blank well containing no enzyme, is used for calculating the ratio between the activities of Factor VII polypeptide and wild-type Factor VIIa:

Ratio=(A405 nm Factor VII polypeptide)/(A405 nm Factor VIIa wild-type).

Based thereon, Factor VII polypeptides with an activity lower than, comparable to, or higher than native Factor VIIa may be identified, such as, for example, Factor VII polypeptides where the ratio between the activity of the Factor VII polypeptide and the activity of native Factor VII (wild-type FVII) is about 1.0 versus above 1.0.

The activity measured in this assay may sometimes be referred to as “amidolytic activity”.

The activity of the Factor VII polypeptides may also be measured using a physiological substrate such as Factor X (“In Vitro Proteolysis Assay”), suitably at a concentration of 100-1000 nM, where the Factor Xa generated is measured after the addition of a suitable chromogenic substrate (eg. S-2765). In addition, the activity assay may be run at physiological temperature.

In Vitro Proteolysis Assay (Assay 2)

Native (wild-type) Factor VIIa and Factor VII polypeptide (both hereinafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 μM) in 100 μL 50 mM HEPES, pH 7.4, containing 0.1 M NaCl, 5 mM CaCl₂ and 1 mg/mL bovine serum albumin, are incubated for 15 min. Factor X cleavage is then stopped by the addition of 50 μL 50 mM HEPES, pH 7.4, containing 0.1 M NaCl, 20 mM EDTA and 1 mg/mL bovine serum albumin. The amount of Factor Xa generated is measured by the addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used for calculating the ratio between the proteolytic activities of Factor VII polypeptide and wild-type Factor VIIa:

Ratio=(A405 nm Factor VII polypeptide)/(A405 nm Factor VIIa wild-type).

Based thereon, a Factor VII polypeptide with an activity lower than, comparable to, or higher than native Factor VIIa may be identified, such as, for example, Factor VII polypeptides where the ratio between the activity of the Factor VII polypeptide and the activity of native Factor VII (wild-type FVII) is about 1.0 versus above 1.0.

One-Stage Coagulation Assay (Clot Assay) (Assay 4)

The clot assay was used to assess the ability of Factor VIIa polypeptides to make blood clot. Factor VII polypeptides may also be assayed for specific activities (“clot activity”) by using a one-stage coagulation assay. For this purpose, the sample to be tested is diluted in 50 mM PIPES-buffer (pH 7.2), 1% BSA and 40 μl is incubated with 40 μl of Factor VII deficient plasma and 80 μl of human recombinant tissue factor containing 10 mM Ca²⁺ and synthetic phospholipids. Coagulation times (clotting times) are measured and compared to a standard curve using a reference standard in a parallel line assay.

Factor X Activation (Assay 5)

Factor VII polypeptides may be assayed for their ability to activate coagulation factor X by using an activation assay (Assay 5). For this purpose, lipidated TF (10 pM) and the sample to be tested is diluted to a concentration of 100 μM in BSA buffer (see assay 4) and incubated 60 min at room temperature before Factor X (50 nM) is added. The reaction is stopped after another 10 min by addition of ½ volume stopping buffer (50 mM Hepes, pH 7.4, 100 mM NaCl, 20 mM EDTA). The amount of Factor Xa generated is determined by adding substrate S2765 (0.6 mM, Chromogenix, and measuring absorbance at 405 nm continuously for 10 min.

High Molecular Weight Protein (HMWP) Content

A size-exclusion HPLC method was used to determine the relative content of high molecular weight proteins (HMWP) in 40K-PEG-rFVIIa formulations.

Determination of HMWP Content—HMWP GPC Method

SE-HPLC, size exclusion chromatography method was used for analysing the HMWP content of samples under non-dissociating conditions.

The column used was Tosoh Bioscience TSKgel G4000SWXL or column with similar specifications. The analytical run is performed by isocratic elution at 21-25° C., followed by UV-detection at 215 nm. The eluent buffer contained 25 mM Bis-Tris propane, 10 mM calcium acetate, 20% isopropanol, buffered to pH 6.8. Normal run time for a sample was 40 minutes.

A chromatogram typically consists of two minor peaks followed by two major peaks. With the shortest retention two minor peaks appear—the polymer peak with the lowest retention, followed by a peak corresponding to the monomer of di-pegylated FVIIa and the dimer of mono-pegylated FVIIa. These are followed by to major peaks: the monomer of mono-pegylated FVIIa and the salt peak.

Heavy Chain Fragmentation Assay

For the purpose of determining the content of heavy chain fragmentation products, a reverse phase HPLC was run on an ACE 3 μm C4, 300 Å, 4.6×100 mm column (Advanced Chromatography Technologies, part. no. ACE-213-1046). Column temperature: 60° C. A-buffer: 0.05% v/v trifluoracetic acid. B-buffer: 0.06% v/v trifluoracetic acid, 80% v/v acetonitrile. Denaturation buffer: 6M Guanidine hydrochloride, 50 mM Tris, 5 mM calcium chloride, pH 7.5. Samples were prepared from 50 μl analysis sample+50 μl denaturation buffer+5 μl DTT+1 μl acetic acid and incubated at 60° C. for 15 min.

The column was eluted with a linear gradient from 35 to 80% B in 30 minutes. Flow rate: 0.7 mL/min. Detection: 214 nm. Load: 25 μg FVIIa. The initial content of heavy chain degradation products was subtracted from the measured content of heavy chain degradation product, i.e. the initial content of heavy chain degradation products was set to 0%. The content of heavy chain degradation products at the time x was then calculated as:

$$\begin{matrix}\% = ( HCDP ( x ) - HCDP ( 0 ) ) / ( HCDP ( x ) - HCDP ( 0 ) + FVII ( x ) ) \times 100 \% \\ = ( HCDP ( x ) - HCDP ( 0 ) ) / ( FVII ( 0 ) ) \times 100 \%\end{matrix}$$

wherein HCDP(x) is the measured content of heavy chain degradation products at time x, HCDP(0) is the measured initial content of heavy chain degradation products, and FVII(x) is the content of the intact Factor VII polypeptide at time x.

Preparation and Purification of Factor VII Polypeptides

Human purified Factor VIIa suitable for use in the present invention is preferably made by DNA recombinant technology, e.g. as described by Hagen et al., Proc. Natl. Acad. Sci. USA 83: 2412-2416, 1986, or as described in European Patent No. 0 200 421 (ZymoGenetics, Inc.).

Factor VII may also be produced by the methods described by Broze and Majerus, J. Biol. Chem. 255 (4): 1242-1247, 1980 and Hedner and Kisiel, J. Clin. Invest. 71: 1836-1841, 1983. These methods yield Factor VII without detectable amounts of other blood coagulation Factors. An even further purified Factor VII preparation may be obtained by including an additional gel filtration as the final purification step. Factor VII is then converted into activated Factor VIIa by known means, e.g. by several different plasma proteins, such as Factor XIIa, IX a or Xa. Alternatively, as described by Bjoern et al. (Research Disclosure, 269 September 1986, pp. 564-565), Factor VII may be activated by passing it through an ion-exchange chromatography column, such as Mono Q® (Pharmacia fine Chemicals) or the like, or by autoactivation in solution.

Factor VII-related polypeptides may be produced by modification of wild-type Factor VII or by recombinant technology. Factor VII-related polypeptides with altered amino acid sequence when compared to wild-type Factor VII may be produced by modifying the nucleic acid sequence encoding wild-type Factor VII either by altering the amino acid codons or by removal of some of the amino acid codons in the nucleic acid encoding the natural Factor VII by known means, e.g. by site-specific mutagenesis.

It will be apparent to those skilled in the art that substitutions can be made outside the regions critical to the function of the Factor VIIa molecule and still result in an active polypeptide. Amino acid residues essential to the activity of the Factor VII polypeptide, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for coagulant, respectively cross-linking activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The introduction of a mutation into the nucleic acid sequence to exchange one nucleotide for another nucleotide may be accomplished by site-directed mutagenesis using any of the methods known in the art. Particularly useful is the procedure that utilizes a super-coiled, double-stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with DpnI which is specific for methylated and hemi-methylated DNA to digest the parental DNA template and to select for mutation-containing synthesized DNA. Other procedures known in the art for creating, identifying and isolating variants may also be used, such as, for example, gene shuffling or phage display techniques.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J. C. Janson and Lars Ryden, editors, VCH Publishers, New York, 1989. Following purification, the preparation preferably contains less than 10% by weight, more preferably less than 5% and most preferably less than 1%, of non-Factor VII polypeptides derived from the host cell.

Factor VII polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII polypeptides may be activated by passing it through an ion-exchange chromatography column, such as Mono Q® (Pharmacia) or the like, or by autoactivation in solution. The resulting activated Factor VII polypeptide may then be formulated and administered as described in the present application.

The following examples illustrate practice of the invention. These examples are included for illustrative purposes only and are not intended in any way to limit the scope of the invention claimed.

### Working Examples

### Example 1

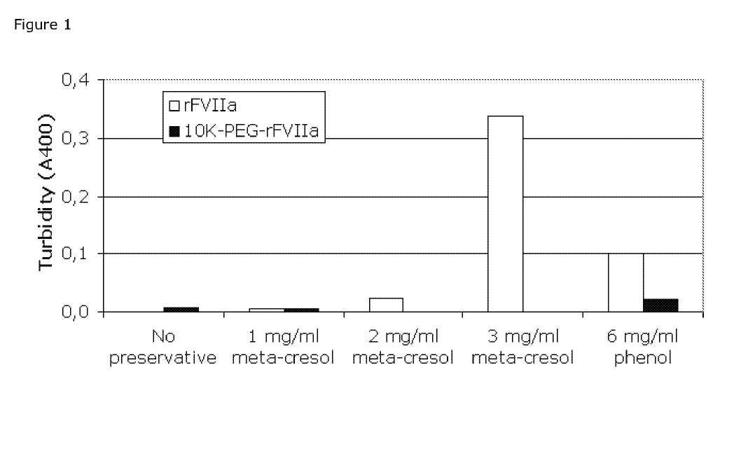

Solutions of rFVIIa and 10K-PEG-rFVIIa were buffer exchanged on NAP10 columns to a buffer containing 6.25 mM CaCl₂ and 6.25 mM histidine at pH 6.0. Samples were then made containing 20 μl of either rFVIIa or 10K-PEG-rFVIIa solution and 5 μl 5 mg/ml m-cresol, 10 mg/ml m-cresol, 15 mg/ml m-cresol or 30 mg/ml phenol. Final concentrations were approximately 2.6 mg/ml rFVIIa or 2.6 mg/ml 10K-PEG-rFVIIa, 5 mM CaCl₂, 5 mM histidine and 1, 2 or 3 mg/ml m-cresol or 6 mg/ml phenol. The samples were shaken (vortexed) briefly and transferred to 15 μl cuvettes with a 1.5 mm light path. The turbidity was then measured as absorbance at 400 nm. High turbidity is a sign of precipitation of large aggregates of the FVII polypeptide. It was seen from the experiment that the solutions of rFVIIa show significant turbidity in the presence of preservatives, indicating that m-cresol and phenol induce precipitation of the sample (see FIG. 1). On the other hand, the solution of 10K-PEG-rFVIIa shows low turbidity in the presence of preservatives, indicating that this molecule remains soluble (see FIG. 1).

This example demonstrates that functionalisation with one or more polyethylene glycol (PEG) moieties increases the solubility of FVIIa in a liquid formulation that contains an aromatic preservative.

### Example 2

40K-PEG-rFVIIa was formulated at 22 mg/ml with 10 mM His, pH 5.5, 20 mM CaCl2, 6% sucrose and 0.5 mg/ml methionine. Samples of 200 μl were freeze-dried and reconstituted in either 3 mg/ml m-cresol or 15 mg/ml benzyl alcohol. Immediately after reconstitution samples of 20 μl were withdrawn and diluted to 1 mg/ml in 10 mM His, pH 5.5, 20 mM CaCl2, 6% sucrose. These reference samples with low preservative content were then stored at 4° C. The samples containing 3 mg/ml m-cresol and 15 mg/ml benzyl alcohol were also stored at 4° C. 5 weeks later, another 20 μl was withdrawn and diluted to 1 mg/ml. These samples, together with the reference samples were then assayed by size-exclusion chromatography, clot activity and FX activation. Table 1 shows the analysis results, with the clot activity and FX activation given relative to the values obtained for the reference samples. These results show that the presence of high concentration of preservatives results in little or no degradation of the samples,

TABLE 1

| | 3 mg/ml m-cresol | 15 mg/ml benzyl alcohol |
|---|---|---|
| Clot activity after 5 weeks | 102% | 98% |
| (% of reference) | | |
| FX activation after 5 weeks | 100% | 95% |
| (% of reference) | | |
| Dimer/2-PEG + HMWP | 3.3% | 3.5% |
| (reference) | | |
| Dimer/2-PEG + HMWP | 3.1% | 3.5% |
| after 5 weeks | | |

### Example 3

A series of different formulations of glycopegylated 40K-PEG-rFVIIa were prepared. All formulations contained 20 mg/ml 40K-PEG-rFVIIa, 10 mM histidine, 10 mg/ml sucrose, 25 mg/ml mannitol, 0.07 mg/ml tween80 and 0.5 mg/ml methionine. The concentration of 40K-PEG-rFVIIa is, in this example, specified as the protein content, without taking the PEG group into account. In addition, the formulations had the conditions and components specified in table 2, as follows:

TABLE 2

| Sample No. | Reconstitution liquid |
|---|---|
| 1 | pH 5.75, 10 mM CaCl₂ |
| 2 | pH 5.75, 10 mM CaCl₂, 5 mg/ml phenol |
| 3 | pH 5.75, 100 mM CaCl₂ |
| 4 | pH 5.75, 100 mM CaCl₂, 5 mg/ml |
| | phenol |
| 5 | pH 5.5, 35 mM CaCl₂, 3 mg/ml phenol |
| 6 | pH 5.25, 10 mM CaCl₂ |
| 7 | pH 5.25, 10 mM CaCl₂, 5 mg/ml phenol |
| 8 | pH 5.25, 100 mM CaCl₂ |
| 9 | pH 5.25, 100 mM CaCl₂, 5 mg/ml phenol |

Two samples of each formulation were prepared and incubated at 5° C. At 0, 4, 8 and 12 weeks, samples were visually inspected for [FVII polypeptide] precipitation and two aliquots were withdrawn from each sample, diluted to a 40K-PEG-rFVIIa concentration of 1 mg/ml and frozen at −80° C. After all samples had been collected, one sample from each formulation and time point was analysed for fragmentation of the FVII polypeptide, high molecular weight protein and dimer/2-PEG. All four samples from each formulation and time point were assayed for amidolytic activity. For formulation 2 and 9, all four samples from all time points were assayed for clot activity

Table 3 shows the change in the content of FVII polypeptide fragments (Δfragment), High Molecular Weight Protein (ΔHMWP) and dimer/2-PEG (Δdimer), as well as the result of visual inspection after 4 and 12 weeks.

TABLE 3

| For- | | | | Visual 4 | Visual 12 |
|---|---|---|---|---|---|
| mulation | Δfragment | ΔHMWP | Δdimer | weeks | weeks |
| 1 | 17.3% | −0.2% | −0.2% | Clear | Clear |
| 2 | 8.3% | 0.0% | 0.3% | Clear | Clear |
| 3 | 14.9% | −0.1% | −0.1% | Clear | Clear |
| 4 | 6.6% | 0.2% | 0.8% | Precipitated | Precipitated |
| 5 | 7.0% | 0.1% | 0.1% | Clear | Precipitated |
| 6 | 5.8% | 0.0% | 0.2% | Clear | Precipitated |
| 7 | 1.4% | 0.0% | 0.5% | Clear | Precipitated |
| 8 | 5.5% | 0.4% | 0.8% | Clear | Clear |
| 9 | 2.2% | 0.3% | 1.6% | Clear | Clear |

All formulations containing an aromatic preservative are clearly seen to have a lower rate of fragmentation than the otherwise identical formulation without a preservative (formulation 2 versus 1, formulation 4 versus 3, formulation 7 versus 6, formulation 9 versus 8). On the other hand, the presence or absence of an aromatic preservative does not seem to affect FVII polypeptide precipitation.

1. A liquid, aqueous pharmaceutical composition comprising:
(i) a Factor VII(a) polypeptide functionalised with one or more polyethylene glycol (PEG) moiety, wherein each of the one or more PEG moieties has a molecular weight in the range of of 5,000-50,000 Da;
(ii) a buffering agent suitable for keeping pH in a range of about 5.0 to about 9.0; and
(iii) at least one aromatic preservative in a concentration of at least 0.1 mg/mL.

2. The composition of claim 1, wherein each of the one or more PEG moieties has a molecular weight in the range of 35,000-45,000 Da.

3. The composition of claim 1, wherein at least one of the one or more PEG moieties is glycopegylated.

4. The composition of claim 1, wherein the at least one aromatic preservative is selected from the group consisting of meta-cresol, phenol, benzyl alcohol, chlorocresol, and combinations thereof.

5. The composition of claim 1, wherein the at least one aromatic preservative has a concentration of 0.1-30.0 mg/mL.

6. The composition of claim 5, wherein said aromatic preservative is selected from the group consisting of 1.0-5.0 mg/mL meta-cresol, 1.0-10.0 mg/mL phenol, 5.0-30.0 mg/mL benzyl alcohol, and 1.0-5.0 mg/mL chlorocresol.

7. The composition according to claim 1, wherein the Factor VII(a) polypeptide has a concentration of 0.1-90 mg/mL.

8. The composition of claim 1, which has a pH value selected from the group consisting of from about 5.0 to about 8.0, from about 5.0 to about 7.5; from about 5.0 to about 7.0; from about 5.0 to about 6.5, from about 5.0 to about 6.0, from about 5.5 to about 7.0; from about 5.5 to about 6.5, from about 6.0 to about 7.0, from about 6.4 to about 6.6, and from about 5.2 to about 5.7.

9. The composition of claim 1, wherein the buffering agent comprises at least one component selected from the group consisting of acids and salts of MES, PIPES, ACES, BES, TES, HEPES, TRIS, histidine, imidazole, glycine, glycylglycine, glycinamide, phosphoric acid, acetic acid, lactic acid, glutaric acid, citric acid, tartaric acid, malic acid, maleic acid, and succinic acid.

10. The composition of claim 1, wherein the concentration of the buffering agent is 1-100 mM.

11. The composition of claim 1, wherein the Factor VII(a) polypeptide is 40K-PEG-rFVIIa, wherein the buffering agent keeps the pH within the range of about 5 to about 6 and wherein the at least one aromatic preservative is either phenol in a concentration of 1.0-10.0 mg/ml or m-cresol in a concentration of 1.0-5.0 mg/mL.

12. The composition of claim 1, wherein the Factor VII(a) polypeptide is 40K-PEG-rFVIIa, wherein the buffering agent keeps the pH within the range of about 5 to about 6 and wherein the at least one aromatic preservative is a combination of phenol and m-cresol.

13. A liquid, aqueous pharmaceutical composition comprising the composition of claim 1.

14. (canceled)

15. A method for treating a Factor VII(a)-responsive disorder, the method comprising administering to a subject in need thereof an effective amount of the liquid, aqueous pharmaceutical composition of claim 13.
